(19) United States
(12) Patent Application Publication
(10) Pub. No.: US 2017/0281657 A1
(43) Pub. Date: Oct. 5, 2017

(54) Low Osmolality Gel Composition

(71) Applicant: ImQuest BioSciences, Inc., Frederick, MD (US)

(72) Inventors: Robert W. Buckheit, JR., Myersville, MD (US); Anthony Sang Won Ham, Germantown, MD (US)

(21) Appl. No.: 15/478,942

(22) Filed: Apr. 4, 2017

Related U.S. Application Data
(60) Provisional application No. 62/318,000, filed on Apr. 4, 2016.

(51) Int. Cl. A61K 31/685; A61K 47/38; A61K 9/06; A61K 47/32; A61K 9/00; A61K 47/10; A61K 31/513

(52) U.S. Cl. CPC A61K 31/685; A61K 47/10; A61K 47/38; A61K 31/513; A61K 47/32; A61K 9/0031; A61K 9/06

(57) ABSTRACT

The present invention relates to polymer gel compositions and more specifically to polymer gel compositions having low osmolality for use on rectal mucosa. The present invention extends to their methods of manufacture and use, including as drug delivery vehicles and personal lubricants.

### CROSS REFERENCE TO RELATED APPLICATIONS

This application claims the benefit of U.S. provisional patent application 62/318,000, filed Apr. 4, 2016 the contents of which are hereby incorporated by reference in their entirety.

### STATEMENT OF FEDERALLY FUNDED RESEARCH

This invention was made with U.S. Government support funded by NIH grant U19AI101961. The government has certain rights in this invention.

### FIELD OF THE INVENTION

The present invention relates to low osmolality gel compositions, as well as methods for their manufacture and use for the prevention and/or treatment of diseases and disorders Packaged kits of the compositions and articles of manufacture including the compositions also are provided. Advantageously, the compositions of the present invention are designed for greater compatibility with rectal mucosa.

### BACKGROUND OF THE INVENTION

Various personal lubricants are known to the art, intended to improve lubrication and comfort during sex. By composition, these products are water-based, oil-based or silicone-based. The majority of the compositions actually used today are water-based compositions formulated for vaginal use.

Various microbicides are also known in the art, intended for the prevention of sexually transmitted diseases, including HIV. While condoms are highly effective in preventing the transmission of sexually transmitted diseases, the decision to use condoms is more often (worldwide) a decision made by males than females. Microbicides offer the opportunity for women to take more control of their sexual health. One study estimated that microbicide use could prevent about 2.5 million HIV infections within 3 years, assuming a microbicide that only worked 60% of the time and was used by only 20% of women, in 73 low income countries. Currently, microbicide delivery systems under development include gels, rings, films, and suppositories. Of these systems, vaginal gels remain the preferred choice for the first line of microbicide product development.

Anal sex is common among men who have sex with men (MSM), and practiced by women around the world. There are significant physiological differences between the rectum and the vagina. Structurally, the vagina is composed of a stratified squamous epithelium, while a simple columnar epithelium covers the rectum/lower gastrointestinal tract. Additionally, the length of the colon as an “open cavity” compared to the “closed cavity” of the vagina provides a greater surface area of infection. With reference to pH, the normal vagina is acidic (pH 4-4.5) due to the presence of lactic acid producing Lactobacilli, whereas the rectum has neutral to slightly alkaline pH. Other differences have also been noted in the literature.

There is a need in the art for personal lubricant compositions, microbicides and drug delivery vehicles formulated specifically for the physiology of the rectum. What is also needed are personal lubricant compositions, microbicides and drug delivery vehicles that take into account the pH and relative osmolality of the compartments such that deviations from this range would make the products not amenable for vaginal tissue, rectal tissue and spermatozoa viability.

### SUMMARY OF THE INVENTION

The invention relates to low osmolality gel compositions including methods of their manufacture and use. Advantageously, the gel compositions of the present invention are suitable for use in the rectum as well as other body parts.

In a first aspect, the invention provides a personal lubricant comprising at least one water soluble polyhydric alcohol and at least one water-soluble polymer derived from cellulose, wherein the personal lubricant has an osmolality between about 200 mOsm/kg and about 500 mOsm/kg and a pH of about 7 or less.

In one embodiment, the personal lubricant has an osmolality between about 200 mOsm/kg and about 400 mOsm/kg. In another embodiment, the personal lubricant has an osmolality between about 200 mOsm/kg and about 350 mOsm/kg. In a further embodiment, the personal lubricant has an osmolality between about 200 mOsm/kg and about 300 mOsm/kg. In yet another embodiment, the personal lubricant has an osmolality between about 200 mOsm/kg and about 280 mOsm/kg. In a still further embodiment, the personal lubricant has an osmolality between about 200 mOsm/kg and about 250 mOsm/kg.

In one embodiment, the personal lubricant has an osmolality of about 200 mOsm/kg, about 220 mOsm/kg, about 240 mOsm/kg, about 260 mOsm/kg, about 280 mOsm/kg, about 300 mOsm/kg, about 320, mOsm/kg, about 350 mOsm/kg, about 360 mOsm/kg, about 380 mOsm/kg or about 400 mOsm/kg.

In one embodiment, the personal lubricant has a pH of between about 5.0 and about 7.0. In another embodiment, the personal lubricant has a pH of between about 5.5 and about 6.5. In a further embodiment, the personal lubricant has a pH of between about 6.0 and about 6.5. In yet another embodiment, the personal lubricant has a pH of between about 5.8 and about 6.2. In a still further embodiment, the personal lubricant has a pH of about 5.0, about 5.5, about 6.0, about 6.5 or about 7.0.

In one embodiment, the personal lubricant has an osmolality between about 200 mOsm/kg and about 400 mOsm/kg and a pH between about 5.5 and about 6.5.

In one embodiment, the personal lubricant has an osmolality between about 200 mOsm/kg and about 400 mOsm/kg and a pH of about 6.0.

In one embodiment, the personal lubricant has an osmolality between about 200 mOsm/kg and about 300 mOsm/kg and a pH between about 5.5 and about 6.5.

In one embodiment, the personal lubricant has an osmolality between about 200 mOsm/kg and about 300 mOsm/kg and a pH of about 6.0.

In one embodiment, the personal lubricant has a viscosity of less than about 10 Pa·s. In another embodiment, the personal lubricant has a viscosity of between about 4 and about 5 Pa·s.

In one embodiment, the personal lubricant has a pH of about 7.0 and contains nanoparticles comprising an acidic agent wherein the nanoparticles dissolve at a pH of between about 5.0 and 6.0 and wherein the acidic agent adjusts the pH of the lubricant to between about 5.0 and 6.0 when the nanoparticles dissolve.

In one embodiment, the water-soluble polyhydric alcohol is glycerol.

In one embodiment, the water-soluble polymer is hydroxyethyl cellulose.

In one embodiment, the water-soluble polyhydric alcohol is glycerol and the water-soluble polymer is hydroxyethyl cellulose.

In one embodiment, the personal lubricant further comprises a preservative.

In one embodiment, the preservative is a paraben. In a particular embodiment, the paraben is methylparaben, propylparaben or a combination thereof.

In one embodiment, the personal lubricant comprises about 1 to about 3 weight percent of the water-soluble polyhydric alcohol, or more particularly about 2 weight percent of the water-soluble polyhydric alcohol. In another embodiment, the personal lubricant comprises about 1 to about 2 weight percent of the water-soluble polymer, or more particularly, about 3 weight percent of the water-soluble polymer.

In a second aspect, the present invention is a method of personal lubrication, comprising applying a personal lubricant composition disclosed herein to a body part in need thereof.

In one embodiment, the body part is a vagina, penis, perianal tissue or anus.

In a third aspect, the present invention provides a microbicide composition comprising at least one water-soluble polyhydric alcohol, at least one water-soluble polymer derived from cellulose and at least one antimicrobial agent, wherein the microbicide composition has an osmolality between about 200 mOsm/kg and about 500 mOsm/kg and a pH of about 7 or less.

In one embodiment, the microbicide composition has an osmolality between about 200 mOsm/kg and about 400 mOsm/kg. In another embodiment, the microbicide composition has an osmolality between about 200 mOsm/kg and about 350 mOsm/kg. In a further embodiment, the microbicide composition has an osmolality between about 200 mOsm/kg and about 300 mOsm/kg. In yet another embodiment, the microbicide composition has an osmolality between about 200 mOsm/kg and about 280 mOsm/kg. In a still further embodiment, the microbicide composition has an osmolality between about 200 mOsm/kg and about 250 mOsm/kg.

In one embodiment, the microbicide composition has an osmolality of about 200 mOsm/kg, about 220 mOsm/kg, about 240 mOsm/kg, about 260 mOsm/kg, about 280 mOsm/kg, about 300 mOsm/kg, about 320, mOsm/kg, about 350 mOsm/kg, about 360 mOsm/kg, about 380 mOsm/kg or about 400 mOsm/kg.

In one embodiment, the microbicide composition has a pH of between about 5.0 and about 7.0. In another embodiment, the microbicide composition has a pH of between about 5.5 and about 6.5. In a further embodiment, the microbicide composition has a pH of between about 6.0 and about 6.5. In yet another embodiment, the microbicide composition has a pH of between about 5.8 and about 6.2. In a still further embodiment, the microbicide composition has a pH of about 5.0, about 5.5, about 6.0, about 6.5 or about 7.0.

In one embodiment, the microbicide composition has an osmolality between about 200 mOsm/kg and about 400 mOsm/kg and a pH between about 5.5 and about 6.5.

In one embodiment, the microbicide composition has an osmolality between about 200 mOsm/kg and about 400 mOsm/kg and a pH of about 6.0.

In one embodiment, the microbicide composition has an osmolality between about 200 mOsm/kg and about 300 mOsm/kg and a pH between about 5.5 and about 6.5.

In one embodiment, the microbicide composition has an osmolality between about 200 mOsm/kg and about 300 mOsm/kg and a pH of about 6.0.

In one embodiment, the microbicide composition has a viscosity of less than about 10 Pa·s. In one embodiment, the microbicide composition has a viscosity of between about 4 and about 5 Pa·s.

In one embodiment, the water-soluble polyhydric alcohol is glycerol.

In one embodiment, the water-soluble polymer is hydroxyethyl cellulose.

In one embodiment, the water soluble polyhydric alcohol is glycerol and the water-soluble polymer is hydroxyethyl cellulose.

In one embodiment, the microbicide composition comprises about 1 to about 3 weight percent of the water-soluble polyhydric alcohol, or more particularly about 2 weight percent of the water-soluble polyhydric alcohol. In another embodiment, the microbicide composition comprises about 1 to about 2 weight percent of the water-soluble polymer, or more particularly, about 3 weight percent of the water-soluble polymer.

In one embodiment, the at least one antimicrobial agent is selected from the group consisting of anti-viral agents, anti-bacterial agents, anti-fungal agents or combinations thereof.

In one embodiment, the at least one antimicrobial agent is an anti-viral agent.

In a particular embodiment, the anti-viral agent is selected from the group consisting of protease inhibitors (PIs), nucleoside reverse transcriptase inhibitors (NRTIs), nucleotide reverse transcriptase inhibitors (NtRTIs), non-nucleoside reverse transcriptase inhibitors (NNRTIs), integrase inhibitors, entry inhibitors, maturation inhibitors and pharmaceutically-acceptable salts and precursors thereof.

In one embodiment, the anti-viral agent is a non-nucleoside reverse transcriptase inhibitor.

In one embodiment, the anti-viral agent is a nucleotide reverse transcriptase inhibitor.

In a particular embodiment, the anti-viral agent is tenofovir.

In a particular embodiment, the anti-viral agent is IQP-0528.

In one embodiment, the microbicide composition comprises at least two antimicrobial agents.

In one embodiment, the microbicide composition comprises at least three antimicrobial agents.

In one embodiment, the microbicide composition comprises at least two anti-viral agents, wherein the at two anti-viral agents comprise a non-nucleoside reverse transcriptase inhibitor and a nucleotide reverse transcriptase inhibitor.

In one embodiment, the microbicide composition further comprises a preservative.

In one embodiment, the preservative is a paraben. In a particular embodiment, the paraben is methylparaben, polyparaben or a combination thereof.

In one embodiment, the microbicide composition further comprises a rheology modifier. In a particular embodiment, the rheology modifier is Carbopol®.

In one embodiment, the microbicide composition further comprises a chelator agent. In one embodiment, the chelator agent is ethylene diamine tetraacetic acid (EDTA).

In one embodiment, the microbicide composition further comprises lactic acid.

In one embodiment, the microbicide composition comprises about 1 to about 3 weight percent of the water-soluble polyhydric alcohol, or more particularly about 2 weight percent of the water-soluble polyhydric alcohol.

In one embodiment, the microbicide composition comprises about 1 to about 2 weight percent of the water-soluble polymer, or more particularly, about 3 weight percent of the water-soluble polymer.

In one embodiment, the microbicide composition comprises about 1 to about 2 weight percent of the at least one antimicrobial agent.

In one embodiment, the microbicide composition further comprises at least one non-antimicrobial therapeutic agent. In a particular embodiment, the at least one non-antimicrobial therapeutic agent is a contraceptive agent.

In a fourth aspect, the present invention is a method of preventing a sexually transmitted disease, comprising applying a microbicide composition disclosed herein to a body part in need thereof.

In a fifth aspect, the present invention is a rectal drug delivery vehicle, comprising at least one water-soluble polyhydric alcohol, a water-soluble polymer derived from cellulose and at least one antimicrobial agent, wherein the drug delivery vehicle has an osmolality between about 200 mOsm/kg and about 500 mOsm/kg and a pH of about 7 or less.

In a sixth aspect, the present invention is a pharmaceutical composition comprising at least one water-soluble polyhydric alcohol, at least one water-soluble polymer derived from cellulose and at least one therapeutic agent, wherein the pharmaceutical composition has an osmolality between about 200 mOsm/kg and about 500 mOsm/kg and a pH of about 7 or less.

In one embodiment, the at least one therapeutic agent is selected from a buffering agent, a contraceptive agent, a anticonvulsants, non-narcotic analgesics and non-steroidal anti-inflammatory agents, hypnosedatives and anaesthetics, strong analgesics, theophylline and derivatives, corticosteroids, antibacterial agents, thiazinamium, promethazine, hyoscine-N-butyl-bromide, streptokinase, progesterone, ergotamine tartrate and levodopa.

In one embodiment, the body part is a vagina, penis, perianal tissue or anus.

In a seventh aspect, the invention provides a method of manufacturing the compositions described herein, comprising the steps of:)
(i) preparing an aqueous buffered solution and separating into a first and second portion,
(ii) combining polyhydric alcohol and preservatives in a vial, heating to dissolution and cooling to room temperature,
(iii) adding the mixture from step (ii) to the first portion of buffered solution while stirring,
(iv) adding a volume of the second portion of buffered solution to the vial from step (ii), mixing and adding the volume to the stilling first portion of the buffered solution and repeating step (iv) with all of the second portion of buffered solution
(v) gradually adding water-soluble cellulosic polymer to buffered solution and continue stirring until homogeneous, and
(vi) adjusting pH to about 5.0 to 7.0.

In one embodiment, the at least one preservative compound is selected from methylparaben, propylparaben or combinations thereof.

In one embodiment, the buffered solution is a phosphate buffered solution.

In one embodiment, the polyhydric alcohol is glycerol.

In one embodiment, the cellulosic polymer is hydroxyethyl cellulose.

In one embodiment, the pH is adjusted to about 6.0.

In one embodiment, EDTA is added to the phosphate buffered solution in step (i).

In one embodiment, lactic acid is added to the phosphate buffered solution in step (i).

In one embodiment, a microbicide is added to the phosphate buffered solution in step (i).

In one embodiment, a microbicide is added to the polyhydric alcohol mixture in step (ii).

### BRIEF DESCRIPTION OF DRAWINGS

FIG. 1 provides a graph showing length travel by varying volumes of FID4012 gel over time.

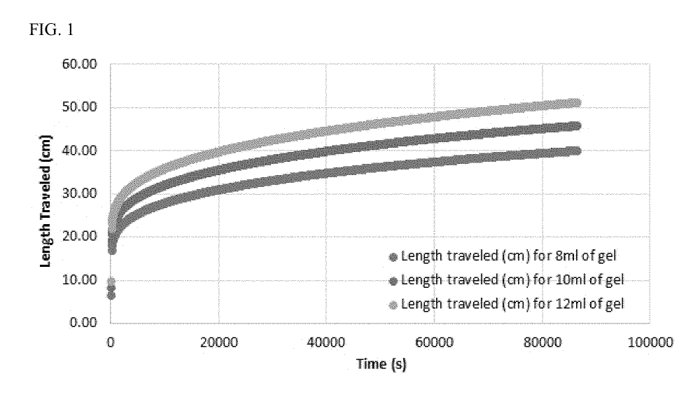

FIG. 2 provides a graph showing ectocervical viability (A) and colorectal viability (B) after 24 exposure to multi-drug DuoGel formulation.

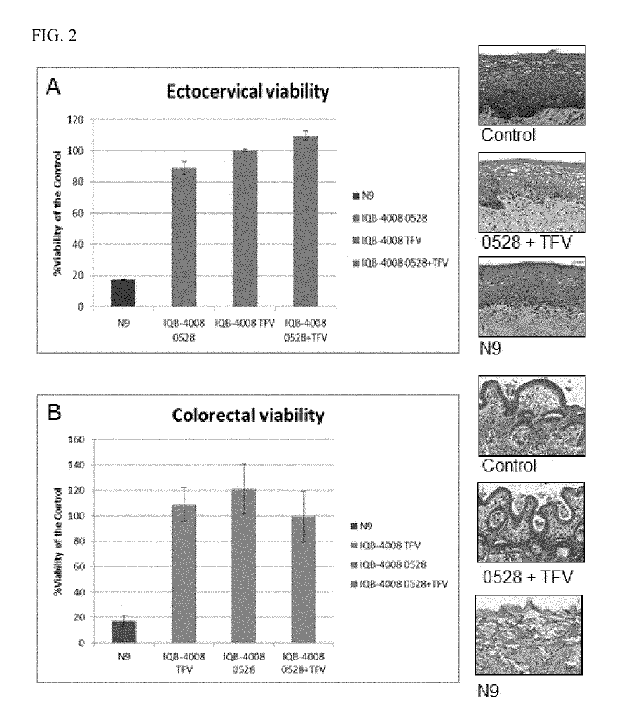

FIG. 3 provides a graph showing ectocervical efficacy and colonic efficacy of DuoGel formulation.

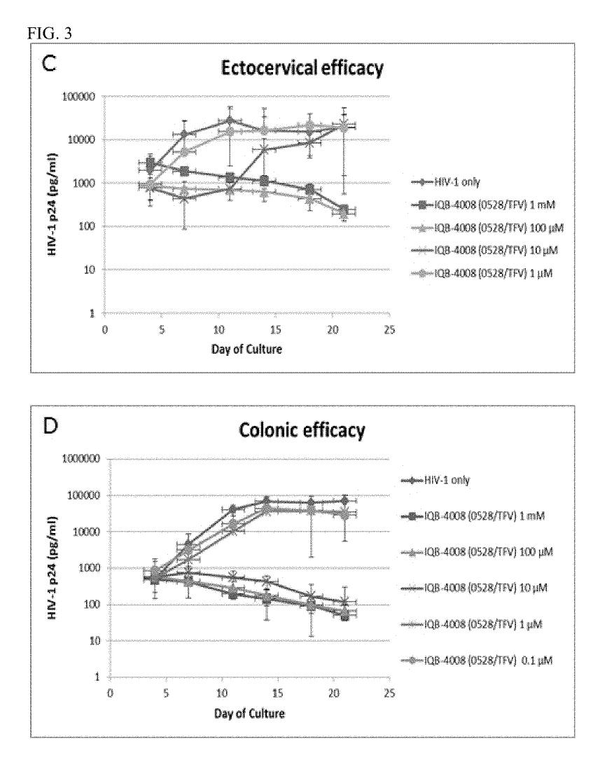

### DETAILED DESCRIPTION OF THE INVENTION

The present invention relates to polymer gel compositions for use as personal lubricants, microbicides and drug delivery vehicles. More specifically the present invention relates to gel compositions having low osmolality for improved safety profiles for use on rectal mucosa. For vaginal use, the low osmolality product is amenable to the integrity of spermatozoa. Sperm are immobilized and killed as osmolality increases form normal physiologic up to 550 mOsm. The low osmolality lubricants described herein protect sperm and thus are amenable to fertilization if desired. In certain embodiments, the polymer gel composition includes at least on therapeutic agent, such as an antimicrobial agent. In one aspect, provided herein is a topical formulation suitable for application to the skin or a mucous membrane of a subject,

### I. Definitions

The term “antimicrobial agent” as used herein refers to a substances (e.g., a drug or chemical) that kills or inhibits the growth of a microbe. Antimicrobial agents are typically classified by the type of microbe they primarily impact. Representative, non-limiting examples of antimicrobial agents include antibacterial agents, anti-viral agents, anti-fungal agents, anti-protozoal and anti-parasitic agents. In addition to pharmaceutical agents, a wide range of chemical and natural compounds can be used as antimicrobial agents.

The term “bioavailability” is the degree to which the pharmaceutically active agent becomes available to the target tissue after the agent's introduction into the body. Enhancement of the bioavailability of a pharmaceutically active agent can provide a more efficient and effective treatment for patients because, for a given dose, more of the pharmaceutically active agent will be available at the targeted tissue sites.

The term “chelating agent” as used herein should be understood to encompass one or more agents which complex and segregate residual traces of free multivalent cations susceptible to cause the physical degradation of the gel matrix (thereby causing loss of viscosity and breakdown of the formulation).

The term “mucosa” or “mucosal tissue” or “mucosal membrane” as used herein interchangeably should be understood to encompass any moist anatomical membrane or surface on a mammal that can be permeated without swallowing.

The term “osmolality” as used herein refers to the concentration of a solution expressed in terms of osmoles of solute per kilogram of solvent (osmol/kg or Osm/kg). Osmolality can be measured by any suitable method, for example, by vapor pressure or freezing point depression.

Vapor pressure osmometers are used to determine the concentration of osmotically active particles that reduce the vapor pressure of the solution, while freezing point osmometers determine the osmotic strength of solution by utilizing freezing point depression.

As used herein, the term “paraben” refers to an ester of p-hydroxybenzoic acid, generally used as a preservative. For example, methylparaben (or methyl paraben) is the methyl ester of p-hydroxybenzoic acid, and is described in the Merck Index (e.g., see Merck Index, 12th Edition, entry 6182 (1996)). Methylparaben is also known as methyl-4-hydroxybenzoate and has the CAS Registry Number 99-76-3. Ethylparaben (or ethyl paraben) is the ethyl ester of p-hydroxybenzoic acid, and is described in the Merck Index (e.g., see Merck Index, 12th Edition, entry 3883 (1996)). Ethylparaben is also known as ethyl-4-hydroxybenzoate and has the CAS Registry Number 120-47-8. Propylparaben (or propyl paraben) is the propyl ester of p-hydroxybenzoic acid, and is described in the Merck Index (e.g., see Merck Index, 12th Edition, entry 8051 (1996)). Propylparaben is also known as propyl-4-hydroxybenzoate and has the CAS Registry Number 120-47-8. Butylparaben (or butyl paraben) is the butyl ester of p-hydroxybenzoic acid, and is described in the Merck Index (e.g., see Merck Index, 12th Edition, entry 1619 (1996)). Butylparaben is also known as butyl-4-hydroxybenzoate and has the CAS Registry Number 94-26-8. Isobutylparaben (or isobutyl paraben) is the isobutyl ester of p-hydroxybenzoic acid. Isobutylparaben is also known as isobutyl-4-hydroxybenzoate and has the CAS Registry Number 4247-02-3. Benzylparaben (or benzyl paraben) is the benzyl ester of p-hydroxybenzoic acid. Benzylparaben is also known as benzyl-4-hydroxybenzoate and has the CAS Registry Number 94-18-8. The term “parabens” includes one or a combination of p-hydroxybenzoic acid esters and salts thereof, including sodium and potassium salts of the benzoate ester.

As used herein, the term “personal lubricant” refers to a composition suitable for providing lubrication during intimate contact, in connection with the insertion of catheters, colostomy bags and similar medical devices, during ultrasound or similar procedure needing improved skin conduction and surface lubrication to prevent chafing.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Such suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

The term “prevent,” “preventing,” or “prevention,” as used herein refers to any reduction, no matter how slight, of a subject's predisposition or risk for developing a condition, disease, disorder or symptom thereof. For purposes of prevention, the subject is any subject, and preferably is a subject that is at risk for, or is predisposed to, developing a condition, disease, disorder. The term “prevention” includes either preventing the onset of a clinically evident condition, disease, disorder altogether or preventing the onset of a pre-clinically evident condition, disease, disorder in individuals at risk. This includes prophylactic treatment of subjects at risk of developing condition, disease, disorder.

The term “polymer” “polymers,” “polymeric,” and similar terms are used in their ordinary sense as understood by one skilled in the art, and thus may be used herein to refer to or describe a large molecule (or group of such molecules) that contains recurring units. Polymers may be formed in various ways, including by polymerizing monomers and/or by chemically modifying one or more recurring units of a precursor polymer. A polymer may be a “homopolymer” comprising substantially identical recurring units formed by, e.g., polymerizing a particular monomer. A polymer may also be a “copolymer” comprising two or more different recurring units formed by, e.g., copolymerizing two or more different monomers, and/or by chemically modifying one or more recurring units of a precursor polymer.

The term “prodrug” as used herein refers to any compound that when administered to a biological system generates the drug substance, i.e. active ingredient, as a result of spontaneous chemical reaction(s), enzyme catalyzed chemical reaction(s), and/or metabolic chemical reaction(s).

The term “prodrug moiety” means a labile functional group which separates from the active inhibitory compound during metabolism, systemically, inside a cell, by hydrolysis, enzymatic cleavage, or by some other process (Bundgaard, Hans, “Design and Application of Prodrugs” in Textbook of Drug Design and Development (1991), P. Krogsgaard-Larsen and H. Bundgaard, Eds. Harwood Academic Publishers, pp. 113-491). Prodrug moieties can serve to enhance solubility, absorption and lipophilicity to optimize drug delivery, bioavailability and efficacy. A “prodrug” is thus a covalently modified analog of a therapeutically-active compound.

As used herein, the term “sexually transmitted disease” is used interchangeably with “STD,” “sexually transmitted infection,” “STI” and/or the plural thereof. An STD is an illness or pathophysiological condition that has a significant probability of transmission between humans by means of any form of sexual contact, including kissing. The term STD may also encompass a person who is infected, and may potentially infect others, without showing signs of disease or infection.

As used herein, the phrases an “effective amount” or a “therapeutically effective amount” of an active agent or ingredient, or pharmaceutically active agent or ingredient, refer to an amount of the pharmaceutically active agent sufficient enough to have an intended effect (e.g., prophylactic, therapeutic) effect upon administration. Effective amounts of the pharmaceutically active agent will vary with the kind of pharmaceutically active agent chosen, the particular condition or conditions being treated, the severity of the condition, the duration of the treatment, the specific components of the composition being used, and like factors.

As used herein, the term “solvent” refers to any pharmaceutically acceptable medium which is a liquid at ambient temperature, in which one or more solutes can be dissolved, or one or more substances can be partially dissolved or suspended.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of symptoms associated therewith, provide improvement to a patient or subject's quality of life, or delay or inhibit the onset of a disease, disorder, or condition.

As used herein, all percentages are by weight of the total composition (i.e., wt %), unless otherwise specified.

The term “viscosity” as used herein refers to the resistance to flow of a material.

Any concentration ranges, percentage range, or ratio range recited herein are to be understood as expressly disclosing and including any concentrations, percentages or ratios of any integer within that range and fractions thereof, such as one tenth and one hundredth of an integer, and any sub-range falling within a range, unless otherwise indicated.

Any number range recited herein relating to any physical feature, including for example, polymer subunits, size or thickness, are to be understood as expressly disclosing and including any integer or fraction of an integer within a disclosed range, or any sub-range within a disclosed range, unless otherwise indicated.

### II. Compositions

The present invention provides gel compositions suitable for use as personal lubricants as well as drug delivery vehicles for therapeutic agents, including antimicrobial agents as well as other therapeutic agents. The present invention extends to pharmaceutical compositions themselves, i.e., the gel composition containing the antimicrobial agent or other drug. Advantageously, the compositions of the present invention are formulated for use on rectal mucosa and are particularly suited for RAI.

### A. Properties

The compositions of the present invention have a suitable osmolality for their intended use, such as rectal use. In exemplary embodiments, the compositions of the present invention have an osmolality of less than about 500 mOsm/kg, less than about 450 mOsm/kg, less than about 400 mOsm/kg, less than about 350 mOsm/kg, less than about 300 mOsm/kg, less than about 250 ms/kg, or less than about 200 mOsm/kg, but each case greater than zero.

In exemplary embodiments, the compositions of the present invention have an osmolality between about 150 mOsm/kg and about 500 mOsm/kg, about 200 mOsm/kg and about 400 mOsm/kg, about 200 mOsm/kg and about 300 mOsm/kg. In exemplary embodiments, the compositions of the present invention have an osmolality of between about 160 mOsm/kg and about 180 mOsm/kg, about 180 mOsm/kg and about 200 mOsm/kg, about 200 and about 220 mOsm/kg, about 240 mOsm/kg and about 260 mOsm/kg, about 260 mOsm/kg and about 280 mOsm/kg, about 280 mOsm/kg and about 300 mOsm/kg, about 300 mOsm/kg and about 320 mOsm/kg, about 320 mOsm/kg and about 340 mOsm/kg, about 340 mOsm/kg and about 360 mOsm/kg, about 360 mOsm/kg and about 380 mOsm/kg, about 380 mOsm/kg and about 400 mOsm/kg, about 400 mOsm/kg and about 420 mOsm/kg, about 420 mOsm/kg and about 440 mOsm/kg, about 440 mOsm/kg and about 460 mOsm/kg, about 460 mOsm/kg and about 480 mOsm/kg, or about 480 mOsm/kg and about 500 mOsm/kg,

In exemplary embodiments, the compositions of the present invention have an osmolality of about 160 mOsm/kg, about 180 mOsm/kg, about 200 mOsm/kg, about 220 mOsm/kg, about 240 mOsm/kg, about 260 mOsm/kg, about 280 mOsm/kg, about 300 mOsm/kg, about 320 mOsm/kg, about 340 mOsm/kg, about 360 mOsm/kg, about 380 mOsm/kg, about 400 mOsm/kg.

### B. pH.

In exemplary embodiments, the compositions of the present invention have a suitable pH for their intended use, such as rectal use. The pH may be adjusted and/or maintained with the aid of acids, bases buffers and other pH-adjusting agents, as is well-known in the art. For example, potassium hydroxide or another alkali metal or alkaline earth metal base may be useful to provide the appropriate pH. Any other physiologically acceptable base may also be used in this manner to adjust the pH from acidic to more neutral.

In exemplary embodiments, the compositions of the present invention have a pH of less than about 7.5, less than about 7.4, less than about 7.3, less than about 7.2, less than about 7.1, less than about 7.0, less than about 6.9, less than about 6.8, less than about 6.7, less than about 6.6, less than about 6.5, less than about 6.4, less than about 6.3, less than about 6.2, less than about 6.1, less than about 6.0, less than about 5.9, less than about 5.8, less than about 5.7, less than about 5.6, less than about 5.5, less than about 5.4, less than about 5.3, less than about 5.2, less than about 5.1, less than about 5.0, less than about 4.9, less than about 4.8, less than about 4.7, less than about 4.6, less than about 4.5, less than about 4.4, less than about 4.3, less than about 4.2, less than about 4.1 or less than about 4.0, but in each case, greater than zero.

In exemplary embodiments, the compositions of the present invention have a pH of between about 4 and about 7.5, about 4.5 and about 7.0, about 5 and about 7.0 or about 5.5 and about 6.5.

In exemplary embodiments, the compositions of the present invention have a pH of about 4, about 4.2, about 4.4, about 4.6, about 4.8, about 5.0, about 5.2, about 5.4, about 5.6, about 5.8, about 6.0, about 6.2, about 6.4, about 6.8, about 7.0, about 7.2, or about 7.4.

### C. Viscosity

In exemplary embodiments, the compositions of the present invention have an appropriate viscosity for adhesion to mucous membranes and a low coefficient of friction. Methods of measuring viscosity are known in the art.

At this viscosity, the composition is capable of adhering to mucous membranes and thereby has a long-lasting effect. In a particular embodiment, the present compositions can have a viscosity, at 25° C., in a range of about 0.050 Pa·s to about 5 Pa·s or about 10 Pa·s or more. In situations where the lubricant composition is designed for use as a stand-alone personal lubricant or as a lubricant for a condom, for example, a packaged lubricated condom product, viscosities (at 25° C.) of less than about 10 Pa·s or less than about 6 Pa·s or less than about 5 Pa·s, but in each case, greater than zero are advantageously useful.

In exemplary embodiments, the compositions of the present invention have a viscosity of about less than about 10 Pa·s.(@1/shear), less than about 9 Pa·s, less than about 8 Pa·s, less than about 7 Pa·s, less than about 6 Pa·s, less than about 5 Pa·s., or less than about 4 Pa·s, but in each case, greater than zero.

In exemplary embodiments, the compositions of the present invention have a viscosity between about 4 and about 5 Pa·s, or more particularly, about 4.0 Pa·s, about 4.1 Pa·s, about 4.2 Pa·s, about 4.3 Pa·s, about 4.4 Pa·s, about 4.5 Pa·s, about 4.6 Pa·s, about 4.7 Pa·s, about 4.8 Pa·s, about 4.9 Pa·s, or about 5.0 Pa·s.

In a particular embodiment, the present invention is a personal lubricant composition having an osmolality of between about 200 mOsm/kg and about 340 mOsm/kg.

In a particular embodiment, the present invention is a personal lubricant composition having an osmolality of between about 220 mOsm/kg and about 320 mOsm/kg.

In a particular embodiment, the present invention is a personal lubricant composition having an osmolality of between about 240 mOsm/kg and about 300 mOsm/kg.

In a particular embodiment, the present invention is a personal lubricant composition having an osmolality of between about 260 mOsm/kg and about 280 mOsm/kg.

In a particular embodiment, the present invention is a personal lubricant composition having an osmolality of about 280 mOsm/kg.

In a particular embodiment, the present invention is a personal lubricant composition having an osmolality between about 200 mOsm/kg and about 500 mOsm/kg and a pH of between about 5.0 and about 7.0.

In a particular embodiment, the present invention is a personal lubricant composition having an osmolality between about 200 mOsm/kg and about 500 mOsm/kg and a pH of between about 5.5 and about 6.5.

In a particular embodiment, the present invention is a personal lubricant composition having an osmolality between about 200 mOsm/kg and about 500 mOsm/kg and a pH of about 6.

In a particular embodiment, the present invention is a personal lubricant composition having an osmolality of between about 200 mOsm/kg and about 300 mOsm/kg having a pH between about 6 and about 7.

In a particular embodiment, the present invention is a personal lubricant composition having an osmolality of between about 200 mOsm/kg and about 300 mOsm/kg having a pH between about 6 and about 6.5.

In a particular embodiment, the present invention is a personal lubricant composition having an osmolality between about 200 mOsm/kg and about 500 mOsm/kg, a pH of between about 5.0 and about 7.0 and a viscosity of less than about 10 Pa·s, or more particularly, between about 4 and about 5 Pa·s.

In a particular embodiment, the present invention is a personal lubricant composition having an osmolality between about 200 mOsm/kg and about 500 mOsm/kg, a pH of between about 5.5 and about 6.5 and a viscosity of less than 10 Pa·s, or more particularly, between about 4 and about 5 Pa·s.

In a particular embodiment, the present invention is a personal lubricant composition having an osmolality between about 200 mOsm/kg and about 500 mOsm/kg, a pH of about 6 and and a viscosity of less than 10 Pa·s, or more particularly, between about 4 and about 5 Pa·s.

In a particular embodiment, the present invention is a microbicide composition comprising at least on therapeutic agent (e.g., an anti-viral agent) and having an osmolality of between about 200 mOsm/kg and about 340 mOsm/kg and comprising at least one antimicrobial agent (e.g., an anti-viral agent).

In a particular embodiment, the present invention is a microbicide composition comprising at least one antimicrobial agent (e.g., an anti-viral agent) and having an osmolality of between about 220 mOsm/kg and about 320 mOsm/kg and comprising at least one antimicrobial agent (e.g., an anti-viral agent).

In a particular embodiment, the present invention is a microbicide composition comprising at least one antimicrobial agent (e.g., an anti-viral agent) and having an osmolality of between about 240 mOsm/kg and about 300 mOsm/kg.

In a particular embodiment, the present invention is a microbicide composition comprising at least one antimicrobial agent (e.g., an anti-viral agent) and having an osmolality of between about 260 mOsm/kg and about 280 mOsm/kg.

In a particular embodiment, the present invention is a microbicide composition comprising at least one antimicrobial agent (e.g., an anti-viral agent) and having an osmolality of about 280 mOsm/kg.

In a particular embodiment, the present invention is a microbicide composition comprising at least one antimicrobial agent (e.g., an anti-viral agent) and having an osmolality between about 200 mOsm/kg and about 500 mOsm/kg and a pH of between about 5.0 and about 7.0.

In a particular embodiment, the present invention is a microbicide composition comprising at least one antimicrobial agent (e.g., an anti-viral agent) and having an osmolality between about 200 mOsm/kg and about 500 mOsm/kg and a pH of between about 5.5 and about 6.5.

In a particular embodiment, the present invention is a microbicide composition comprising at least one antimicrobial agent (e.g., an anti-viral agent) and having an osmolality between about 200 mOsm/kg and about 500 mOsm/kg and a pH of about 6.

In a particular embodiment, the present invention is a microbicide composition comprising at least one antimicrobial agent (e.g., an anti-viral agent) and having an osmolality of between about 200 mOsm/kg and about 300 mOsm/kg having a pH between about 6 and about 7.

In a particular embodiment, the present invention is a microbicide composition comprising at least one antimicrobial agent (e.g., an anti-viral agent) having an osmolality of between about 200 mOsm/kg and about 300 mOsm/kg having a pH between about 6 and about 6.5.

In a particular embodiment, the present invention is a microbicide composition comprising at least one antimicrobial agent (e.g., an anti-viral agent) and having an osmolality between about 200 mOsm/kg and about 500 mOsm/kg, a pH of between about 5.0 and about 7.0 and a viscosity of less than about 10 Pa·s, or more particularly, between about 4 and about 5 Pa·s.

In a particular embodiment, the present invention is a microbicide composition comprising at least one antimicrobial agent (e.g., an anti-viral agent) and having an osmolality between about 200 mOsm/kg and about 500 mOsm/kg, a pH of between about 5.5 and about 6.5 and a viscosity of less than 10 Pa·s, or more particularly, between about 4 and about 5 Pa·s.

In a particular embodiment, the present invention is a microbicide composition comprising at least one antimicrobial agent (e.g., an anti-viral agent) and personal lubricant having an osmolality between about 200 mOsm/kg and about 500 mOsm/kg, a pH of about 6 and a viscosity of less than 10 Pa·s, or more particularly, between about 4 and about 5 Pa·s.

In one embodiment, the present invention is a rectal drug delivery vehicle having an osmolality of between about 200 mOsm/kg and about 340 mOsm/kg.

In one embodiment, the present invention is a rectal pharmaceutical composition comprising at least one therapeutic agent and having a an osmolality of between about 200 mOsm/kg and about 340 mOsm/kg.

### D. Components

The compositions of the present are based on water-soluble (hydrophilic) polymers. As used herein the phrase “water- soluble polymer” and variants thereof refer to a polymer that is at least partially soluble in water, and desirably fully or predominantly soluble in water, or absorbs water. In exemplary embodiments, the water-soluble polymer is derived from cellulose, i.e., a cellulosic polymers. In exemplary embodiments, the cellulosic polymer is hydroxalkyl cellulose having a lower alkyl moiety, such as ethyl, propyl, butyl or the like.

Non-limiting water-soluble polymers suitable for use in the present invention include polyethylene oxide (PEO), pullulan, hydroxypropylmethyl cellulose (HPMC), hydroxyethyl cellulose (HEC), hydroxypropyl cellulose, methyl cellulose, polyvinyl pyrrolidone, carboxymethyl cellulose, sodium carboxy methyl ceullose, ethyl cellulose, polyvinyl alcohol, sodium alginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof.

In one embodiment, the water-soluble polymer is hydroxypropyl methyl cellulose (HPMC).

In another embodiment, the water-soluble polymer is hydroxyethyl cellulose (HEC). It may be used alone or in combination with another cellulosic polymer. In a preferred embodiment, HEC is at least 50% by weight, at least 70% by weight or at least 90% by weight of the cellulosic polymer in the composition. In exemplary embodiments, the cellulosic polymer consists or consists essentially of HEC or, in other words, is substantially entirely (i.e., at least 95% by weight) or entirely HEC. In these embodiments, the HEC is typically present in the composition at a concentration that is at least 1.0%, more typically about least 1.5%. The average molecular weight of the preferred HEC is typically at least 720,000 Da, more typically about 1,000,000 Da. In one embodiment, the average molecular weight of the HEC is between about 700 kDa and about 1200 kDa. In another embodiment, the average molecular weight of the HEC is between about 900 kDa and about1100 kDa.

In exemplary embodiments, high-purity cosmetic grade of hydroxyethyl cellulose (R-grade).

In exemplary embodiments, the water-soluble polymer is present in an amount between about 0.1 to about 10 weight per cent of the final composition. Most preferably, the water-soluble polymer is present in an amount between about 0.5 and about 3% by weight of the composition, or more particularly about 1.5% by weight of the composition.

In certain embodiments, the water-soluble gel polymer matrix may, optionally, contain certain additional polymers such as polyvinyl pyrrolidone and carboxy-functional polymer. The polyvinyl pyrrolidone has a molecular weight of about 10,000 to about 1,200,000. Carboxy-functional polymers suitable for use in a bioadhesive polymeric system include polyacrylic acid, carboxymethyl cellulose, and polymethylacrylic acid. These carboxy-functional polymers have a molecular weight of from about 90,000 to about 1,200,000. The composition may contain from about 0.1 to about 10%, preferably from about 0.2 to about 2%, by weight, of the polyvinyl pyrrolidone-carboxy functional polymer moiety. Most preferably, it should be about 0.45% by weight of the composition. The weight ratio of polyvinyl pyrrolidone to carboxy functional polymer is within the range of about 0.01:1 to about 5:1. When carboxymethyl cellulose is employed as the carboxy-functional polymer, the weight ratio of polyvinyl pyrrolidone to carboxymethyl cellulose is within the range of about 0.01:1 to about 4:1, preferably about 0.5:1 to about 2:1 and most preferably about 1:1.

The compositions of the present invention include a substantial amount of water, either in the form of pure water, or in the form of an aqueous buffer. Typically, the amount of water included in the gel will be less than about 99% by weight, more typically less than about 97% by weight, and in some specific embodiments in the range of about 95% to about 96% by weight.

In exemplary embodiments, compositions of this invention contain at least one polyhydric alcohol which is water-soluble. In one embodiment, the polyhydric alcohol is selected from glycerin, propylene glycol, sorbitol or a combination thereof. In another embodiment, the polyhydric alcohol is polyethylene glycol ranging from molecular weight of from about 300 to about 1450.

In certain embodiments, the composition contains two or more polyhydric alcohols.

In certain embodiments, the composition contains two or more polyhydric alcohols and one or more cellulose gums.

The polyhydric alcohol portion of the product should make up from about 1 to about 10% by weight of the composition. More preferably, the compositions of this invention should contain a combination of two or more polyhydric alcohols and one or more cellulose gums.

An inorganic base may be used to adjust the pH of the composition. Potassium hydroxide or another alkali metal or alkaline earth metal base may be useful to provide the appropriate pH. Of course, any other physiological acceptable base may also be utilized in this manner. From about 0.05 to about 5.0% by weight inorganic base is preferably used.

Optionally, a preservative may be important for use in the products of this invention, in order to preserve the stability of the compositions of this invention and to prevent the growth of microorganisms therein. The preservative portion of the compositions of this invention may be one or more known preservatives, such as methylparaben, benzoic acid, sorbic acid, gallic acid or propylparaben. From about 0.05% to about 0.75% by weight preservative should be used.

In certain embodiments, the compositions of the present invention are microbicide compositions, i.e., contain one or more antimicrobial agents. In exemplary embodiments, the one or more antimicrobial agent is an anti-viral, anti-bacterial, anti-fungal agent, anti-parasitic agent or combination thereof. In exemplary embodiments, the composition may contain from about 0.01% to about 60% of the one or more therapeutic agents, on a weight to weight basis. In certain embodiments, the composition may contain from about 0.10 to about 10.0%, about 1.0 to about 5.0%, about 1.0 to about 3.0%, or about 1.0% of the one or more therapeutic agents.

In an exemplary embodiment, the composition of the present invention is a microbicide that contains one or more therapeutic agents that kill or neutralize a virus and more particularly, a sexually transmitted virus. Representative, non-limiting viral agents include Human Immunodeficiency virus (HIV), Herpes Simplex Virus Types 1 and 2, Human Papilloma Virus, Zika virus, Hepatitis B or Hepatitis C.

In one embodiment, the microbicide comprises one or more anti-viral agents selected from protease inhibitors (PIs), nucleoside reverse transcriptase inhibitors (NRTIs), nucleotide reverse transcriptase inhibitors (NtRTIs), non-nucleoside reverse transcriptase inhibitors (NNRTIs), integrase inhibitors, entry inhibitors, maturation inhibitors and pharmaceutically-acceptable salts and precursors thereof.

In one embodiment, the anti-viral compound may be one or more selected from aciclovir, docosanol, edoxudine, famciclovir, foscarnet, idoxuridine, penciclovir, trifluridine, tromantidine, valaciclovir and vidarabine (all of which treat infection caused by one or more herpes viruses); adefovir, boceprevir, entecavir, ribavirin and taribavirin (all of which treat infection caused by one or more hepatitis viruses); or amantadine, arbidol, oseltamivir, peramivir, rimantidine and zanamivir (all of which treat infection cause by one or more influenza viruses).

In one embodiment, the anti-viral compound may be one or more selected from amprenavir, atazanavir, darunavir, fosamprenavir, indinavir, lopinavir, nelfinavir, ritonavir, saquinavir and tipranavir (all of which are protease inhibitors); abacavir (ABC), amdoxovir, apricitabine (ATC), didanosine (ddl), elvucitabine, emtricitabine (FTC), entecavir (INN), lamivudine (3TC), racivir, stampidine, stavudine (d4T), zalcitabine (ddC) and zidovudine (AZT) (all of which are NRTIs); adefovir (also known as bis-POM PMPA) and tenofovir (both of which are NtRTIs); delavirdine, efavirenz, etravirine, lersivirine, loviride, nevirapine and rilpivirine (all of which are NNRTIs); elvitegravir, globoidnan A, GSK-572, MK-2048 and raltegravir (all of which are integrase inhibitors); enfuviritide, ibalizumab, maraviroc and vicriviroc (all of which are fusion/entry inhibitors); bevirimat and vivecon (both of which are maturation inhibitors); and pharmaceutically-acceptable salts and precursors thereof, and mixtures thereof.

In one particular embodiment, the anti-viral agent is IQP-0528. IQP-0528 is a non-nucleoside reverse transcriptase inhibitor that also blocks virus entry.

In an exemplary embodiment, the composition contains two or more anti-viral agents. In a particular embodiment, the composition contains IPQ-0528 and tenofovir.

In an exemplary embodiment, the composition contains three or more anti-viral agents.

In an exemplary embodiment, the composition of the present invention is a microbicide that contains one or more therapeutic agents that kill or neutralize bacteria and more particularly, a sexually transmitted bacteria. Representative, non-limiting bacterial agents include Chlamydia trachomatis, Neisseria gonorrhoea or Treponema pallidum.

in one embodiment, the microbicide comprises one or more antibiotic agents. In certain embodiments, the antibiotic agent is a beta lactam. In a particular embodiment, the beta lactam is a penam, cephem, carbapenem, monobactam or beta-lactamase inhibitor.

In one embodiment, the microbicide comprises one or more antibiotic agents selected from azithromycin, doxycycline, erythromycin, ofloxacin, cefixime (Suprax), ceftriaxone, ciprofloxacin, trimethoprim/sulfamethoxazole.

In other embodiments, the microbicide comprises one or more antibiotic agents selected from metronidazole, clindamycin, tinidazole, ornidazole, secnidazole, refaximin, trospectomycin, purpuromycin and their pharmaceutically acceptable salts and the like.

Still other embodiments of the compositions of this invention are compositions containing antibacterial agents. The antimicrobial agents may preferably include, but are not limited to, chlorohexidine gluconate, sodium polystyrene sulfonate, sodium cellulose sulfate, silver particles of micro- and sub-micrometer sizes, silver salts and other antibactetial agents known to the art.

In an exemplary embodiment, the composition comprises two or more antibiotic agents.

In an exemplary embodiment, the composition of the present invention is a microbicide that contains one or more therapeutic agents that kill or neutralize fungus and more particularly, a sexually transmitted fungus.

In one embodiment, the agent is an antifungal agents which may include, but are not limited to imidazole compounds such as an azole or imidazole, including but not limited to, miconazole, econazole, terconazole, saperconazole, itraconazole, butaconazole, clotrimazole, tioconazole, fluconazole and ketoconazole, vericonazole, fenticonazole, sertaconazole, posaconazole, bifonazole, oxiconazole, sulconazole, elubiol, vorconazole, isoconazole, flutrimazole and their pharmaceutically acceptable salts and the like. Other antifungal agents may include an allylamine or one from other chemical families, including but not limited to, ternafine, naftifine, amorolfine, butenafine, ciclopirox, griseofulvin, undecyclenic acid, haloprogin, tolnaftate, nystatin, iodine, rilopirox, BAY 108888, purpuromycin and their pharmaceutically acceptable salts.

In an exemplary embodiment, the composition is suitable for use as a drug delivery vehicle for a therapeutic agent that is not a microbicide—either alone or in combination with a microbicide.

In one embodiment, the composition of the present invention is used to deliver buffering agents such as, but not limited to phosphate, citrate, succinate, bicarbonate and in combinations of carboxylates such as fumarate, tartarate, lactate and maleate to adjust the pH of the membranes in order to promote healthy environments.

In one embodiment, the buffering agent is delivered with a microbicide. In another embodiment, the buffering agent is delivered without a microbicide.

In one embodiment, the composition of the present invention may include one or more contraceptive agents. Representative, non-limiting contraceptive agents include nonoxynol-9, octoxynol-9 and menfegol. From about 2 to about 20% contraceptives may be present in the compositions of this invention.

In one embodiment, the contraceptive agent is delivered with a microbicide. In another embodiment, the contraceptive agent is delivered without a microbicide.

In another embodiment, the composition may include one or more analgesics and/or nonsteroidal anti-inflammatory agents. The analgesics and nonsteroidal anti-inflammatory agents may preferably include, but are not limited to, aspirin, ibuprofen, indomethacin, phenylbutazone, bromfenac, fenamate, sulindac, nabumetone, ketorolac, and naproxen and the like.

In one embodiment, the analgesic and/or nonsteroidal anti-inflammatory agent is delivered with a microbicide, In another embodiment, the analgesic and/or nonsteroidal anti-inflammatory agent is delivered without a microbicide.

In another embodiment, the composition may include one or more local anesthetics. Such as benzocaine, lidocaine, dibucaine, benzyl alcohol, camphor, resorcinol, menthol and diphenylhydramine hydrochloride and the like.

In one embodiment, the anesthetic is delivered with a microbicide. In another embodiment, the anesthetic is delivered without a microbicide.

In exemplary embodiments, the composition of the present invention includes one or more therapeutic agents that are intended to have a systemic effect but rectal delivery offers a preferred route of administration. This might occur in several situations: (i) when administration by the oral route results in intolerance, nausea, vomiting or gastric pain; (ii) when patients are uncooperative or have decreased consciousness; (iii) when access to the intravenous route is difficult, e.g. in children or in patients in intensive care units needing multiple drugs and continuous fluid infusions but with few veins undamaged; (iv) in ambulatory patients, when repeated, painful intramuscular administration of drugs is not well accepted

In one embodiment, the composition of the present invention includes one or more of the following: anticonvulsants, non-narcotic analgesics and non-steroidal anti-inflammatory agents, hypnosedatives and anaesthetics, strong analgesics, theophylline and derivatives, corticosteroids, antibacterial agents, thiazinamium, promethazine, hyoscine-N-butyl-bromide, streptokinase, progesterone, ergotamine tartrate and levodopa.

In exemplary embodiments, the composition comprises a solubilizer. The solubilizer moiety enables the water-soluble polymer matrix to maintain its integrity when exposed to certain medicaments used in the compositions of this invention. In some instances, addition of medicament to a water-soluble polymer matrix, such as hydroxyalkyl cellulose, tends to destroy the gel matrix and cause its collapse. The addition of a solubilizer substantially prevents the collapse of the gel matrix and permits the gel matrix to maintain its properties.

Preferably,the solubilizer moiety should be a nonionic compound having a hydrophile-lipophile balance (HLB) between about 10 and about 16. Preferably, the solubilizer should be a polyethoxylated compound having a high ethylene oxide (EO) content. The ethylene oxide content is determined in accordance with ASTM Test No. D 4875-88. The EO content should be at least 20 moles. The molecular weight of the solubilizer moiety should be greater than of the medicament moiety. Thus, the solubilizer moiety should have a molecular weight between about 600 and about 5,000, A solubilizer with molecular weight greater than about 5,000 would not be acceptable due to its disproportionate size with regard to the medicament. Furthermore, its HLB would probably be excessively high to be compatible with the medicament. The solubilizer should be relatively close in weight to that of the medicament compound employed.

More preferably, the solubilizer moiety of the composition of this invention is an ethoxylated esters or ethers, or ethoxylated fatty acid derivatives wherein the fatty acid moiety contains between 8 and 16 carbon atoms. Thus, suitable solubilizers include polyethoxylated alkyl ethers, polyethylene glycol sorbitan fatty acid esters, polyethoxylated castor oils and the like. Most preferably, polyethoxylated, hydrogenated castor oils may be used to produce a clear, low-viscosity personal lubricant composition. The polyethoxylated castor oil may be hydrogenated or non-hydrogenated, however, the castor oil is most preferably hydrogenated.

In one embodiment, the gel optionally contains about 0.5 to about 5 percent by weight ethoxylated solubilizer. In some embodiments, any added medicaments might, without a solubilizer moiety, raise the pH of the composition by interfering or reacting with the polymer gel matrix so as to change the pH. Use of a solubilizer provides more control over pH and substantially prevents a medicament from reacting with the polymer gel matrix thereby permitting the hydroxyethyl cellulose to maintain the appropriate pH without the use of additional buffers.

In various embodiments, the ratio of therapeutic agent/medicament to solubilizer is about 5:1 to about 0.5:1. More preferably, the ratio of medicament to solubilizer is about 1.1:1. Preferably, the ratio of medicament to water-soluble polymer matrix is about 5:1 to about 0.5:1. More preferably, the ratio of medicament to water-soluble polymer matrix is about 1:1. Preferably, the ratio of solubilizer to water-soluble polymer matrix is from about 0.5:2 to about 1:1. More preferably, the ratio of solubilizer to water-soluble polymer matrix is about 0.87:1

Deodorants and fragrances useful in the compositions of this invention include sodium bicarbonate, aluminum chloride, aluminum chlorohydrates, aluminum zirconium chlorohydrates, buffered aluminum sulfate, triclosan and trichlorocarbanilide.

The compositions of this invention may also contain pharmaceutically or cosmetically acceptable additives. These additives include stabilizers, preservatives, excipients, binders, vehicles, chelating agents, antioxidants, coloring agents, flavors, odor controlling agents and the like.

In a particular embodiment, the composition may contain include emollients, moisturizers, humectants, pigments, dyes, pearlescent compounds, nacreous pigments, bismuth oxychloride coated mica, titanium dioxide coated mica, colorants, fragrances, biocides, preservatives, alpha hydroxy acids, antioxidants, antiperspirant agents, exfoliants, hormones, enzymes, medicinal compounds, vitamins, salts, electrolytes, alcohols, polyols, polypropylene glycol, polyisobutene, polyoxyethylene, behenic acid, behenyl, sugar-alcohols, absorbing agents for ultraviolet radiation, botanical extracts, surfactants, silicone oils, organic oils, waxes, alkaline or acidic or buffering agents, film formers, thickening agents, hyaluronic acid, fumed silica, hydrated silica, talc, kaolin, starch, modified starch, mica, nylon, clay, bentonite, organo-modified clays and combinations thereof.

Compositions of this invention should remain stable over time without separating into different constituent components. Preferably, the compositions should remain stable for twenty weeks at 30° C., 40° C. or 50° C. or at room temperature for one year.

In an exemplary embodiment, the composition of the present invention is a personal lubricant comprising (i) about 0.5 to about 5 percent by weight of a hydroxyalkyl cellulose water-soluble polymer; (ii) about 1 to about 5 percent by weight polyhydric alcohol; and about (iii) 85 to about 95 percent by weight water, and having a pH of about 5.5 to about 6.5.

In certain embodiment, the personal lubricant has an osmolality between about 200 and about 500 mOsm/kg, or more particularly, between about 200 and about 400 mOsm/kg, and even more particularly, between about 200 and about 300 mOsm/kg.

In a particular embodiment, the personal lubricant of the present invention has the formula shown in Table 1:

TABLE 1
Personal Lubricant Gel IQL-1001

| Reagent | Quantity |
|---|---|
| Water | 96.21% (w/w) |
| Sodium Phosphate, dibasic | 25 mM, pH 6 |
| Sodium Phosphate, monobasic | 25 mM, pH 6 |
| Methylparaben | 0.20% (w/w) |
| Propylparaben | 0.05% (w/w) |
| Glycerol | 2.02% (w/w) |
| Hydroxyethyl cellulose | 1.52% (w/w) |

In an exemplary embodiment, the composition of the present invention is a pharmaceutical composition, comprising (i) about 0.5 to about 5 percent by weight hydroxyalkyl cellulose water-soluble polymer; (ii) about 0.5 to about 5 percent by weight of one or more therapeutic agents (e.g., an antimicrobial agent; (iii) about 1 to about 5 percent by weight polyhydric alcohol; and (iii) about 85 to about 95 percent by weight water, and having a pH of about 5.5 to about 6.5. In certain embodiment, the personal lubricant has an osmolality between about 200 and about 500 mOsm/kg, or more particularly, between about 200 and about 300 mOsm/kg, and even more particularly between about 200 and about 300 mOsm/kg. In a particular embodiment, the pharmaceutical composition is a microbide composition having the formula shown in Table 2, below:

TABLE 2
Microbicide Composition

| Component | Percent Mass (% w/w) |
|---|---|
| Phosphate Buffer Solution (25 mM; pH 6) | 93.90 |
| Glycerol | 2.50 |
| Methylparaben | 0.20 |
| Propylparaben | 0.05 |
| Hydroxyethylcellulose (HEC) | 2.10 |
| Carbopol | 0.25 |
| Anti-viral agent | 1.0 |

Optionally, the composition of the present invention may further comprise one or more In functional agents designed to cause physiological or physical changes in the area to which they are applied. These functional agents range from agents that self-warm when exposed to moisture, e.g. polyols, agents that act on nerve endings to simulate a perceived sensation such as warming, cooling and/or tingling, and agents that could in sufficient quantity increase localized blood flow, e.g. vasodilators.

Non-limiting examples of warming agents that may be added to the composition of the present invention include capsaicin, gingerol, vanillyl ethyl ether, vanillyl propyl ether, vanillyl butyl ether, vanillyl pentyl ether, vanillyl hexyl ether, vanillyl butyl ether acetate, 4-(1-menthoxymethyl)-2-phenyl-1,3-dioxolan, 4-(1-menthoxymethyl)-2-(3′,4′-dihydroxyphenyl)-1,3-dioxolan, 4-(1-menthoxymethyl)-2-(2′-hydroxy-3′-methoxyphenyl)-1,3-dioxolan, 4-(1-menthoxymethyl)-2-(4′-methoxyphenyl)-1,3-dioxolan, 4-(1-menthoxymethyl)-2-(3′,4′-methylenedioxyphenyl)-1,3-dioxolan, 4-(1-menthoxymethyl)-2-(3′-methoxy-4′-hydroxyphenyl)-1,3-dioxolan, red, pepper oil, red pepper oleoresin, ginger oleoresin, nonylic acid vanillyl amide, Spilanthes acmella extract, Zanthoxylum alatum extract, Zanthoxylum piperitum extract, sanshool I, sanshool II, sanshoamide, black pepper extract, chavicine, piperine, or spilanthol.

Non-limiting examples of cooling agents that may be added to the composition of the present invention include menthol, menthone, camphor, pulegol, isopulegol, cineol, mint oil, peppermint oil, spearmint oil, eucalyptus oil, 3-1-menthoxypropane-1,2-diol, N-alkyl-p-menthane-3-carboxamide, 3-1-menthoxy-2-methylpropane-1,2-diol, p-menthane-3,8-diol, 2-1-menthoxyethane-1-ol, 3-1-menthoxpropane-1-ol, 4-1-menthoxybutane-1-ol, 1-(2-hydroxy-4-ethylcyclohexyl)-ethanone, menthyl 3-hydroxybutanoate, menthyl lactate, menthone glycerin ketal, 2-(2-1-menthyloxyethyl)ethanol, menthyl glyoxylate, N-methyl-2,2-isopropylmethyl-3-methylbutanamide, menthyl 2-pyrrolidone-5-carboxylate, monomenthyl succinate, alkali metal salts of monomenthyl succinate, and alkali earth metal salts of monomenthyl succinate, monomenthyl glutarate, alkali metal salts of monomenthyl glutarate, alkali earth metal salts of monomenthyl glutarate, N-[[5-methyl-2-(1-methylethyl) cyclohexyl]carbonyl]glycine, p-menthane-3-carboxylic acid glycerol ester, Menthol propylene glycol carbonate; or menthol ethylene glycol carbonate, and 6-isopropyl-3,9-dimethyl-1,4-dioxaspiro[4.5]decan-2-one.

### III. Method of Use

The compositions of this invention may be used by individuals for personal lubrication or when antimicrobial activity and/or other therapeutic activity is desired. For example, the compositions may be applied to the body externally or internally.

In one method the composition may be used as a personal lubricant for sexual activity. The method can be implemented and/or used by either party to the sexual activity. Thus, one partner could use the present method to protect himself/herself (as well as the partner) with or without the partner's knowledge of the method being used. The method may be used before the sexual activity, or during the sexual activity, or after the sexual activity or a combination thereof. The method includes applying the personal lubricant composition to the skin, the vagina, the penis, the perianal tissue or the anus.

The compositions may be applied digitally or with an applicator to a body part in need thereof, or may be applied to a condom or a diaphragm. The composition may be reapplied as needed for the duration that lubrication is required.

In one embodiment, the present invention is a method of personal lubrication, comprising applying a composition of the present invention to a body part in need thereof. In an exemplary embodiment, provided is a method for lubricating a vaginal, anal or genital surface, comprising spreading about 0.1 mL to about 50 mL, or from about 5 mL to about 25 mL, or from about 10 mL to about 15 mL of the personal lubricant composition provided herein across one or more vaginal, anal or genital surfaces, in a manner that causes the lubricant gel to coat and remain in contact with the vaginal, anal or genital surfaces.

In another embodiment, provided herein is a method of applying a personal lubricant as provided herein onto the skin of a subject, comprising dispensing about 0.1 mL to about 10 mL of the personal lubricant composition provided herein onto the skin, and spreading the lubricant to produce a lubricating effect. In one embodiment, the personal lubricant composition is dispensed into the hand and applied to the skin, the vagina, the penis, the perianal tissue or the anus. In another embodiment, the personal lubricant is dispensed directly into the vagina or anus or onto the penis.

In one embodiment, the present invention is a method of preventing a sexually transmitted disease, comprising applying a composition of the present invention to a body part in need thereof. This method is not intended to reduce the need and/or use of other protective measures (e.g., condoms), but is rather intended to supplement and increase the protection afforded by these other measures. Of course, this method can also be used where other protective measures are not used for any reason or are used improperly

In one embodiment, the present invention is a method of treating a sexually transmitted disease, comprising applying a composition of the present invention to a body part in need thereof.

In one embodiment, the present invention is a method of simultaneously providing contraception and treatment/prevention of a sexually transmitted disease in a female patient.

Where the method involves preventing and/or treating a sexually transmitted disease, the disease may be caused by a virus, bacteria, fungi, parasite or protozoan. In certain embodiments, the method prevents and/or treats more than one sexually transmitted disease (e.g., HIV and HSV).

In one embodiment, the sexually transmitted disease is caused by a virus. In a particular embodiment, the composition of this invention is used to reduce the risk of transmission of HIV/AIDS. HIV can be found in genital secretions, e.g., semen or vaginal fluid, and can be transmitted during sexual intercourse. In addition to HIV, numerous other sexually transmitted diseases are caused by viral infection including, but not limited to, genital herpes (herpes simplex virus), genital warts (human papilloma virus), hepatitis B, hepatitis D, hepatitis A, hepatitis C, hepatitis E and molluscum contagiosum (pox virus).

In one embodiment, the sexually transmitted disease is caused by bacteria. Numerous sexually transmitted disease are caused by bacterial infection, including but not limited to, chancroid (Chancroid (Haemophilus ducreyi), chlamydia (Chlamydia trachomatis), gonorrhea (Neisseria gonorrhea), granuloma inguinale (Calymmatobacterium granulomatis), lymphogranuloma venereum (Chlamydia trachomatis) and syphilis (Treponema pallidum).

In one embodiment, the sexually transmitted disease is caused by fungi. Yeast infections are a representative, non-limiting example of a sexually transmitted disease caused by fungus (Candida albicans).

In one embodiment, the sexually transmitted disease is caused by a protozoan. Trichomoniasis is a representative, non-limiting example of a sexually transmitted disease caused by a parasite (Trichomonas vaginalis).

### IV. Method of Manufacture

The compositions of this invention may be prepared conventionally, or they may be prepared in accordance with the method of preparation of this invention. Conventional preparation consists of dissolving water soluble components such as polyhydric alcohol (e.g. glycerol), chelator, methylparaben, etc. and other preservatives in water and then adding and dissolving the polymer. These methods were intended to achieve the dissolution of cellulose polymer without forming lumps. In one embodiment, these processes are performed under vacuum.

Measurements for osmolality may be performed using any suitable method, for example, using vapor pressure osmometry (Vapro vapor pressure osmometer 5520 Wescor, Inc., Logan, Utah). The device may be calibrated with, for example, using Opti-mole 100, 290, and 1000 mmol/kg osmolality standards. In alternate embodiments, the osmolality may be measuring using freezing point depression osmometry (Advanced Instrumental Model 3250 freezing point osmometer, Norwood, Mass.).

Measurement of pH may be performed using any suitable method, for example, using the Orion 4-Star Plus Benchtop pH/ISE Meter (Thermo Fisher Scientific) with an Orion 8235BN PerpHect Ross flat surface pH probe and calibrated using three points, pH 4.0, 7.0, and 10.0.

A method for preparing a personal lubricant is provided comprising the steps of:
(i) preparing an aqueous buffered solution and separating into a first and second portion,
(ii) combining polyhydric alcohol and preservatives in a vial, heating to dissolution and cooling to room temperature,
(iii) adding the mixture from step (ii) to the first portion of buffered solution while stirring,
(iv) adding a volume of the second portion of buffered solution to the vial from step (ii), mixing and adding the volume to the stirring first portion of the buffered solution and repeating step (iv) with all of the second portion of buffered solution
(v) gradually adding water-soluble cellulosic polymer to buffered solution and continue stirring until homogeneous, and
(vi) adjusting pH to about 5.0 to 7.0.

One aspect of this method that is particularly effective at preparing the lubricant compositions is the repeated mixing of aqueous buffered solution in the vial used to dissolve the polyhydric alcohol and addition to the first buffered solution. Another aspect of this method that is particularly effective at preparing the lubricant compositions is the gradual addition of polymer to the buffered solution mixture to prevent clumping. Furthermore, The order of addition of reagents is a particular aspect of this formulation that contributes to the properties of the compositions described herein.

In one embodiment, the second portion of step a) is ¼, ⅓, ½, ⅔, or ¾ of the prepared aqueous buffered solution.

In one embodiment, at least one antimicrobial agent is added to the first portion buffered solution of step (i).

In one embodiment, tenofovir is added to the first portion buffered solution of step (i).

In one embodiment, at least one chelator agent is added to the first portion buffered solution of step (i).

In one embodiment, at least EDTA is added to the first portion buffered solution of step (i).

In one embodiment, at least one antimicrobial agent is added to the cooled mixture of step (ii).

In one embodiment, IQP-0528 is added to the cooled mixture of step (ii).

In various embodiment, step d) is repeated 2×, 3×, 4×, 5×, 6×, 7×, 8×, 9×, 10×, 11, 12×, 13×, 14×, 15×, or more than 15×.

In one embodiment, the polymer added in step (v) is hydroxyethylcellulose.

In one embodiment, carbopol is added with the polymer in step (v).

In various embodiments, solution in step e) is stirred for about 45-180 minutes, 90-150 minutes, about 90 minutes, about 120 minutes, or about 150 minutes until homogeneous.

In various embodiments, the pH is adjusted to between about 5.5 and 6.5 with 4M sodium hydroxide. In a particular embodiment, the pH is adjusted to about 6.0 with 4M sodium hydroxide.

In one particular embodiment, a protocol is provided as follows:

Manufacturing Protocol #1: Prepare sodium phosphate buffer solution and transfer to first container. Transfer a portion of the solution to a second container and keep the remaining portion in the first container continually stirring.

Combine methylparaben, propylparaben and glycerol in a separate bottle or sealable container. Heat to 100° C. while stirring until parabens are visually dissolved.

Remove from heat and allow paraben/glycerol container to reach room temperature while continually mixing. Transfer mixture to the first container of the stirring Phosphate Buffer Solution.

Add one volume Phosphate Buffer Solution from the second container to the paraben/glycerol bottle, mix vigorously and transfer solution to first container of Phosphate Buffer Solution. Repeat until all Phosphate Buffer Solution from the glass bottle has been mixed in the paraben/glycerol bottle and added to the stirring Phosphate Buffer Solution.

Continue mixing first container of Phosphate Buffer Solution at room temperature for between about 15-90 minutes, between about 30-36 minutes, between about 30-45 minutes, or for about 30 minutes.

Add hydroxyethylcellulose to the mixing solution gradually to minimize clumping. Cover and continue mixing.

Reduce stirring speed and continue mixing until homogeneous and then adjust pH to about 6.0 with 4M sodium hydroxide.

In another embodiment, an antiviral-containing DuoGel gel composition is prepared as follows:

| Component | Percent Mass (% w/w) |
|---|---|
| Phosphate Buffer Solution (25 mM; pH 6) | 93.90 |
| Glycerol | 2.50 |
| Methylparaben | 0.20 |
| Propylparaben | 0.05 |
| Hydroxyethylcellulose (HEC) | 2.10 |
| Carbopol | 0.25 |
| antiviral agent | 1.0 |

In another particular embodiment, a protocol is provided as follows:

Manufacturing Protocol #2: Prepare sodium phosphate buffer solution and transfer to first container. Transfer a portion of the solution to a second container and keep the remaining portion in the first container continually stirring.

Combine methylparaben, propylparaben and glycerol in a separate bottle or sealable container. Heat to 100° C. while stirring until parabens are visually dissolved.

Remove from heat and allow paraben/glycerol container to reach room temperature while continually mixing. Add antiviral agent and mix solution until visually homogeneous paste is obtained. Transfer mixture to the stirring first container of Phosphate Buffer Solution.

Add one volume Phosphate Buffer Solution from the second container to the paraben/glycerol bottle, mix vigorously and transfer solution to Phosphate Buffer Solution. Repeat until all Phosphate Buffer Solution from the second container has been mixed in the paraben/glycerol bottle and added to the first container of stirring Phosphate Buffer Solution.

Continue mixing first container of Phosphate Buffer Solution at room temperature for between about 15-90 minutes, between about 30-36 minutes, between about 30-45 minutes, or for about 30 minutes.

Add hydroxyethylcellulose and carbopol to the first container of stirring Phosphate Buffer Solution to minimize clumping. Cover and continue mixing.

In various embodiments, solution is mixed for about 45-180 minutes, 90-150 minutes, about 90 minutes, about 120 minutes, or about 150 minutes.

Reduce stirring speed and continue mixing until homogeneous and then adjust with sodium hydroxide.

In various embodiments, the pH is adjusted to between about 5.5 and 6.5 with 4M sodium hydroxide. In a particular embodiment, the pH is adjusted to about 6.0 with 4M sodium hydroxide.

In yet another embodiment, a preparation containing dual-antiviral IQP-0528/Tenofovir DuoGel gel composition is prepared as follows:

Tenofovir (TFV) is a nucleotide reverse transcriptase inhibitor and IQP-0528 is a non-nucleoside reverse transcriptase inhibitor that also blocks virus entry. TFV and IQP-0528 alone have shown antiviral activity as microbicide gels. These drugs have been chosen to make a combination microbicide gel containing 1% TFV/1% IQP-0528.

### (i) Components:

| Component | Percent Mass (% w/w) |
|---|---|
| Phosphate Buffer Solution (25 mM; pH 6) | 93.15 |
| Glycerol | 2.50 |
| Methylparaben | 0.20 |
| Propylparaben | 0.05 |
| Hydroxyethylcellulose (HEC) | 1.00 |
| Carbopol | 1.0 |
| EDTA | 0.05 |
| Lactic acid | 0.05 |
| IQP-0528 | 1.0 |
| Tenovir | 1.0 |

In another particular embodiment, a protocol is provided as follows:

Manufacturing Protocol #3: Prepare sodium phosphate buffer solution and transfer to first container. Transfer a portion of the solution to a second container and keep the remaining portion in the first container continually stirring.

Add Tenovir to the first container of stirring Phosphate Buffer Solution.

Add EDTA to the first container of stirring Phosphate Buffer Solution.

Add Lactic acid to the first container of stirring Phosphate Buffer Solution.

Combine methylparaben, propylparaben and glycerol in a separate bottle or sealable container. Heat to 100° C. while stirring until parabens are visually dissolved.

Remove from heat and allow paraben/glycerol container to reach room temperature while continually mixing. Add IQP-0528 and mix solution until visually homogeneous paste is obtained. Transfer mixture to the first container of stirring Phosphate Buffer Solution.

Add one volume Phosphate Buffer Solution from the second container to the paraben/glycerol bottle and mix vigorously and transfer solution to first container of Phosphate Buffer Solution. Repeat until all Phosphate Buffer Solution from the second container has been mixed in the paraben/glycerol bottle and added to the first container of stirring Phosphate Buffer Solution.

Continue mixing first container of Phosphate Buffer Solution at room temperature for between about 15-90 minutes, between about 30-36 minutes, between about 30-45 minutes, or for about 30 minutes.

Add hydroxyethylcellulose and carbopol to the mixing solution gradually to minimize clumping.

In various embodiments, solution is mixed for about 45-180 minutes, 90-150 minutes, about 90 minutes, about 120 minutes, or about 150 minutes.

Reduce stirring speed and continue mixing until homogeneous and then adjust with sodium hydroxide.

In various embodiments, the pH is adjusted to between about 5.5 and 6.5 with 4M sodium hydroxide. In a particular embodiment, the pH is adjusted to about 6.0 with 4M sodium hydroxide.

In a further broad aspect of the invention, methods of making or manufacturing condom products are provided. Such methods comprise: providing a condom; contacting the condom with the bi- or multiphasic, silicone-containing lubricant composition described elsewhere herein, in an amount effective to lubricate the condom for use; placing the lubricated condom in a package.

### V. Articles of Manufacture

In some embodiments, the compositions provided herein are packaged as articles of manufacture containing a packaging material, within the packaging material a personal lubricant composition provided herein and formulations thereof, and a label that indicates the intended use (e.g., personal lubrication, prevention of infection).

The articles of manufacture provided herein include packaging materials. Packaging materials for use in packaging products are well known to those of skill in the art (see, e.g., U.S. Pat. Nos. 5,323,907, 5,052,558 and 5,033,252). Examples of packaging materials include but are not limited to, blister packs, bottles, tubes, vials, jars, containers, foil packets, aerosol bottles and devices, and any packaging material suitable for a selected formulation and intended mode of administration and treatment. A wide array of formulations of the compositions provided herein and formulations thereof are contemplated.

In certain embodiments, the compositions are presented in the form of a unit dosage form, such as a self-contained delivery device, such as a suppository or an encapsulated bead in a gelatin coating, such as is common in the art for distribution of bath oils (e.g., see U.S. Pat. Nos. 5,254,294 and 4,597,885) in a pack or dispenser device, which may include one or more unit dosage forms containing a composition provided herein. The pack may, for example, include metal or plastic foil, such as a blister pack. The pack or dispenser device may be accompanied by instructions for administration. Compositions provided herein also may be prepared, placed in an appropriate container, and labeled for appropriate use, such as application to the genitals.

In another embodiment, provided herein are containers in which the compositions of the subject invention are sold and/or distributed. In one embodiment, these containers include the compositions provided herein and have instructions for their use. In another embodiment, the containers are glass, metal or plastic (or other appropriate inert material). In one embodiment, the formulation is prepared for immediate use. In one embodiment, the instructions for use are written on the outside of the container. In another embodiment, the composition is packaged in a plastic bottle, tube or vial, which includes instructions for use thereof on the outside of the bottle, tube or vial, which includes an easy to open closure, such as a pump-dispenser type device as part of a cap assembly or flip-top closure, that renders it convenient and easy to use during sexual activity.

In some embodiments, the composition is packaged in a watertight tube made of deformable metal or plastic that is sealed at one end and has a removable closure or cap at the other, such as is used to contain and dispense toothpaste. The cap may be a screw-on type that must be removed completely to dispense the contents, or may have a hinged flip-type cap that can be opened without detaching it from the tube. An advantage of the flip-type cap is that is can be easily opened or closed with one hand. The lubricant is dispensed by squeezing the tube.

In another embodiment, the compositions provided herein are packaged in a container equipped with a manually-operated dispensing pump mechanism, such as those known in the art (e.g., see U.S. Pat. Nos. 6,286,732, 6,006,949 and 5,405,057). Such pump mechanisms allows a quantity of the lubricant to be conveniently dispensed when manually operated, such as by depressing a pump mechanism with one hand. In one embodiment, the package is configured to allow direct application of the composition to the body part in need thereof.

In some embodiments, the composition is packaged as a single-use package. In one embodiment, the packets are made of plastic, metal foil, laminates or metallized plastic. In some embodiments, the packet is pre-scored or pre-notched to aid in the opening of the package. In one embodiment, the single use package comprises between about 5 mL to about 25 mL of the personal lubricant disclosed herein.

In another embodiment, the composition is packaged within an applicator as a single-use package. In one embodiment, the applicator is a vaginal applicator (e.g., see U.S. Pat. Nos. D494,676, D320,084, D294,063, D279,504, D266,702) or other device adapted for delivery of a substance to a cavity in the body (e.g., see U.S. Pat. Nos. 6,537,260, 5,531,703 and 4,351,336).

### VI. Kits

In another embodiment, the present invention also provides kits. In various embodiments the kits include a composition of the present invention in a package or other enclosure, instructions for use, and optionally an applicator. In another embodiment, the kit is provided in a wrapping (such as a plastic) that surrounds the kit. In one embodiment, the applicator is provided inside the package. In other embodiments, the packaging is selected from among a cardboard or paper box, a plastic pouch or a foil pouch. Packaging for the formulation is generally not critical, and there are a number of ways in which the personal lubricant composition may be packaged.

In one embodiment, the kit includes a composition provided herein and an applicator for application of the composition. In one embodiment, the applicator is a dropper, a swab, a stick, a pump, a spray or a syringe. In one embodiment, the applicator is a pump dispenser. In another embodiment, the kit includes a compositions provided herein and a prophylactic. In one embodiment, the prophylactic is a condom.

The following examples serve to illustrate the compositions and methods of this invention. However, they are not presented in order to limit the scope of the invention in any way.

### EXAMPLES

### Example 1

### Preparation of Personal Lubricant Composition

A personal lubricant composition was prepared having the formulation shown in Table 1.

TABLE 1
Personal Lubricant Composition

| Component | Percent Mass (% w/w) |
|---|---|
| Phosphate Buffer Solution (25 mM; pH 6) | 96.21 |
| Glycerol | 2.02 |
| Methylparaben | 0.20 |
| Propylparaben | 0.05 |
| hydroxyethylcellulose (HEC) | 1.52 |

Continue mixing at room temperature for 30 minutes.

Add hydroxyethylcellulose was added to the mixing solution gradually over 5 minutes to minimize clumping. The mixing solution and hydroxyethylcellulose was covered and mixed for an additional for 2 hours.

After 2 hours, the stirring speed was reduced until the solution was homogeneous and then pH was adjusted to 6.0 with 4M sodium hydroxide.

### Example 2

### Preparation of IQP-0528 DuoGel Gel Composition

Provided is a gel formulation, designed for safe and efficacious use in both the vagina and rectum, which delivers the nonnucleoside reverse transcriptase inhibitor (IN RTI) IQP-0528 as a topical anti-HIV microbicide. The data presented summarize diverse, pharmacologically relevant evaluations of candidate gels (termed “DuoGel™s”) including their rheological, in vitro and ex vivo safety and bioactivity properties

IQP-0528 is a non-nucleoside reverse transcriptase inhibitor that also blocks virus entry.

| Component | Percent Mass (% w/w) |
|---|---|
| Phosphate Buffer Solution (25 mM; pH 6) | 93.90 |
| Glycerol | 2.50 |
| Methylparaben | 0.20 |
| Propylparaben | 0.05 |
| Hydroxyethylcellulose (HEC) | 2.10 |
| Carbopol | 0.25 |
| IQP-0528 | 1.0 |

Manufacturing Protocol:

One liter of sodium phosphate buffer solution was prepared and transferred to a beaker. Half of the solution was transferred to a glass bottle and the other half was kept continually stirring.

Methylparaben, propylparaben and glycerol were combined in a separate bottle, sealed with a magnetic stir bar and heated to 100° C. while stirring to 450 rpm until parabens were visually dissolved.

The mixture was removed from heat and allowed to reach room temperature while continually mixing. The magnetic stir bar was removed and IQP-0528 was added to the solution and manually mixed with sterile probe until visually homogeneous paste was obtained. The mixture was transferred to the stirring Phosphate Buffer Solution.

One volume of Phosphate Buffer Solution from the glass bottle was added to the paraben/glycerol bottle and mixed via vortexing for 30 seconds and the solution transferred to Phosphate Buffer Solution. This step was repeated until all Phosphate Buffer Solution from the glass bottle was mixed in the paraben/glycerol bottle and added to the stirring Phosphate Buffer Solution.

The stirring phosphate buffered solution was stirred at room temperature for an additional 30 minutes.

Hydroxyethylcellulose and carbopol were added to the mixing solution gradually over 5 minutes to minimize clumping, and then covered and stirred for an additional 2 hours.

After 2 hours, the stirring speed was reduced until the solution was homogeneous and then pH was adjusted to 6.0 with 4M sodium hydroxide.

### Example 3

### Preparation of IQP-0528/Tenovir DuoGel Gel Composition

Tenofovir (TFV) is a nucleotide reverse transcriptase inhibitor and IQP-0528 is a non-nucleoside reverse transcriptase inhibitor that also blocks virus entry. TFV and IQP-0528 alone have shown antiviral activity as microbicide gels. Because combination therapy will likely be more potent than mono-therapy, these drugs have been chosen to make a combination microbicide gel containing 1% TFV/1% IQP-0528.

| Component | Percent Mass (% w/w) |
|---|---|
| Phosphate Buffer Solution (25 mM; pH 6) | 93.15 |
| Glycerol | 2.50 |
| Methylparaben | 0.20 |
| Propylparaben | 0.05 |
| Hydroxyethylcellulose (HEC) | 1.00 |
| Carbopol | 1.00 |
| EDTA | 0.05 |
| Lactic acid | 0.05 |
| IQP-0528 | 1.0 |
| Tenovir | 1.0 |

Manufacturing Protocol:

One liter of sodium phosphate buffer solution was prepared and transferred to a beaker.

Half of the solution was transferred to a glass bottle and the other half was kept continually stirring.

Tenovir was added to the stirring Phosphate Buffer Solution.

EDTA was added to the stirring Phosphate Buffer Solution.

Lactic acid was added to the stirring Phosphate Buffer Solution.

Methylparaben, propylparaben and glycerol were combined in a separate bottle, sealed with a magnetic stir bar and heated to 100° C. while stirring to 450 rpm until parabens were visually dissolved.

The mixture was removed from heat and allowed to reach room temperature while continually mixing. The magnetic stir bar was removed and IQP-0528 was added and manually mixed with sterile probe until a visually homogeneous paste was obtained. The mixture was transferred to the stirring Phosphate Buffer Solution.

One volume Phosphate Buffer Solution from the glass bottle was added to the paraben/glycerol bottle and mixed via vortexing for 30 seconds and transferred to the Phosphate Buffer Solution. This step was repeated until all Phosphate Buffer Solution from the glass bottle was mixed in the paraben/glycerol bottle and added to the stirring Phosphate Buffer Solution.

The Phosphate Buffer Solution was mixed at room temperature for an additional 30 minutes.

Hydroxyethylcellulose and carbopol were added to the mixing solution gradually over 5 minutes to minimize clumping, covered and mixed for an additional 2 hours.

After 2 hours, the stirring speed was reduced until the solution was homogeneous and then pH was adjusted to 6.0 with 4M sodium hydroxide.

### Example 4

### Evaluation of Gel Compositions

Gel compositions were evaluated from physicochemical and biological properties. First, the pH and osmolality of the DuoGels were defined by a target product profile. DuoGel viscosity was measured under parallel plate geometry from 1E-5 to 200 s-1. In vitro drug release was performed in Franz cells through a cellulose membrane over 6 hours. The rheological spreading and distribution of 4 mL of DuoGel was evaluated under 1.143 lbf.

In Vitro Toxicity and Efficacy:

In vitro evaluations: In vitro toxicity of the DuoGels was performed against CaSki, HEC1A, and ME180 cell lines and Lactobacilli for 24 hours. In vitro efficacy was performed in PBMC and TZM-bl against HIV-1 infection for 7 days.

Explant evaluations: The ex vivo toxicity and efficacy of the DuoGels were performed in both polarized explant ectocervical and colorectal tissues. For toxicity evaluations, the biopsied tissue was set in a polarized transwell system and the DuoGel formulation applied for 24 hour culture. Tissue viability was determined via histological analysis (H&E staining). Efficacy was similarly evaluated in the polarized transwell system. Efficacy was evaluated over a 21 day culture with HIV replication being monitored via p24 immunohistochemistry.

No toxicity observed to Ca-Ski, ME180 or HEC1A cells after 24 hours of exposure up to a concentration of 1 mg/mL.

No toxicity to Lactobacillus after 24 hours up to a concentration of 1 mg/mL.

No toxicity to epivaginal tissue after 24 hours up to a concentration of 1 mg/mL.

Ectocervical (A) and colonic (B) tissue viability after 24 hour exposure to the multi-drug DuoGel formulation. Polarized tissue was set up in duplicate and treated with gel and nonoxynol-9 (N9) containing gels. After treatment one of the tissues was processed for the MTT assay and the other was processed for histology. The data represent mean ±SD of 3 independent tissue donors.

DuoGel efficacy evaluation in ectocervical (C) and colonic (D) tissue. DuoGels and HIV-1 were applied to the apical surface of the tissues and cultured overnight. the tissues were washed and remained in culture for 21 days. Culture supernatant was collected every 3 to 4 days and fresh medium replenished. HIV-1 replication was determined using a p24 ELISA (Perkin Elmer). Models of epithelial irritation are available such as the slug mucosal irritation model (Adriaens et al. Sex Transm Dis 35:512-516, 2008) and the rabbit penile irritation model.

DuoGel formulation FID4012 was identified as the lead formulation for the multi-drug vaginal/rectal microbicide gel due to its defined target product profile and in vitro and ex vivo activity.

We claim:

1. A personal lubricant composition comprising at least one water soluble polyhydric alcohol and at least one water-soluble polymer derived from cellulose, wherein the personal lubricant has an osmolality between about 200 mOsm/kg and about 500 mOsm/kg and a pH of about 7 or less.

2. The composition of claim 1, wherein the personal lubricant has an osmolality between about 200 mOsm/kg and about 350 mOsm/kg.

3. The composition of claim 1, wherein the personal lubricant has an osmolality of about 200 mOsm/kg, about 220 mOsm/kg, about 240 mOsm/kg, about 260 mOsm/kg, about 280 mOsm/kg, about 300 mOsm/kg, about 320, mOsm/kg, about 350 mOsm/kg, about 360 mOsm/kg, about 380 mOsm/kg or about 400 mOsm/kg.

4. The composition of claim 1, wherein the personal lubricant has a pH of between about 5.8 and about 6.2.

5. The composition of claim 1, wherein the personal lubricant has an osmolality between about 200 mOsm/kg and about 300 mOsm/kg and a pH between about 5.5 and about 6.5.

6. The composition of claim 1, wherein the personal lubricant has a viscosity of between about 4 and about 5 Pa·s.

7. The composition of claim 1, wherein the water soluble polyhydric alcohol is glycerol and the water-soluble polymer is hydroxyethyl cellulose.

8. The composition of claim 1, wherein the personal lubricant further comprises a paraben preservative.

9. The composition of claim 1, wherein the personal lubricant comprises about 1 to about 3 weight percent of the water-soluble polyhydric alcohol.

10. A method of personal lubrication, comprising applying a personal lubricant composition of claim 1 to a body part in need thereof.

11. A microbicide composition comprising at least one water-soluble polyhydric alcohol, at least one water-soluble polymer derived from cellulose and at least one antimicrobial agent, wherein the microbicide composition has an osmolality between about 200 mOsm/kg and about 500 mOsm/kg and a pH of about 7 or less.

12. The composition of claim 11, wherein the microbicide composition has an osmolality between about 200 mOsm/kg and about 350 mOsm/kg.

13. The composition of claim 11, wherein the microbicide composition has a pH of between about 5.8 and about 6.2.

14. The composition of claim 11, wherein the microbicide composition has an osmolality between about 200 mOsm/kg and about 300 mOsm/kg and a pH between about 5.5 and about 6.5.

15. The composition of claim 11, wherein the microbicide composition has a viscosity of between about 4 and about 5 Pa·s.

16. The composition of claim 11, wherein the at least one antimicrobial agent is selected from the group consisting of anti-viral agents, anti-bacterial agents, anti-fungal agents or combinations thereof.

17. The composition of claim 16, wherein the anti-viral agent is selected from the group consisting of protease inhibitors (PIs), nucleoside reverse transcriptase inhibitors (NRTIs), nucleotide reverse transcriptase inhibitors (NtRTIs), non-nucleoside reverse transcriptase inhibitors (NNRTIs), integrase inhibitors, entry inhibitors, maturation inhibitors and pharmaceutically-acceptable salts and precursors thereof.

18. The composition of claim 16, wherein the anti-viral agent is tenofovir.

19. The composition of claim 16, wherein the anti-viral agent is IQP-0528.

20. The composition of claim 11, wherein the microbicide composition further comprises a rheology modifier Carbopol®.

21. The composition of claim 11, wherein the microbicide composition further comprises a chelator agent.

22. A method of reducing or preventing transmission of a sexually transmitted disease, comprising applying the microbicide composition of claim 11 to a body part in need thereof.

23. A rectal drug delivery vehicle comprising at least one water-soluble polyhydric alcohol, a water-soluble polymer derived from cellulose and at least one antimicrobial agent, wherein the drug delivery vehicle has an osmolality between about 200 mOsm/kg and about 500 mOsm/kg and a pH of about 7 or less.

24. A pharmaceutical composition comprising the composition of claim 1 or 11 and at least one therapeutic agent, wherein the pharmaceutical composition has an osmolality between about 200 mOsm/kg and about 500 mOsm/kg and a pH of about 7 or less.

25. The pharmaceutical composition of claim 24, wherein the at least one therapeutic agent is selected from a buffering agent, a contraceptive agent, a anticonvulsants, non-narcotic analgesics and non-steroidal anti-inflammatory agents, hypnosedatives and anaesthetics, strong analgesics, theophylline and derivatives, corticosteroids, antibacterial agents, thiazinamium, promethazine, hyoscine-N-butyl-bromide, streptokinase, progesterone, ergotamine tartrate, levodopa and combinations thereof.

26. A method of manufacturing a composition, comprising the steps of:
a) preparing an aqueous buffered solution and separating into a first and second portion,
b) combining polyhydric alcohol and preservatives in a vial, heating to dissolution and cooling to room temperature,
c) adding the mixture from step (ii) to the first portion of buffered solution while stirring,
d) adding a volume of the second portion of buffered solution to the vial from step (d), mixing and adding the volume to the stirring first portion of the buffered solution and repeating step (iv) with all of the second portion of buffered solution
e) gradually adding water-soluble cellulosic polymer to buffered solution and continue stirring until homogeneous, and
f) adjusting pH to about 5.0 to 7.0.
